(19) United States
(12) Patent Application Publication
(10) Pub. No.: US 2004/0028620 A1
(43) Pub. Date: Feb. 12, 2004

(54) Separation of living particles from a gas under pressure

(76) Inventors: Andre Monnot, Saint Remy Les Chevreuse (FR); Corinne Nawrocki, Fonbeauzard (FR); Stephane Melen, Saint Crespin S/Moine (FR); Valerie Schreiber, Meudon (FR)

Correspondence Address: Air Liquide, Intellectual Property Department, Suite 1800, 2700 Post Oak Boulevard, Houston, TX, 77056, US

(21) Appl. No.: 10/432,828

(22) PCT Filed: Nov. 21, 2001
(86) PCT No.: PCT/FR01/03662

(30) Foreign Application Priority Data
Nov. 24, 2000 (FR) 00/15209

(51) Int. Cl. A61L 9/04; A61K 9/14

(52) U.S. Cl. 424/46

(57) ABSTRACT

The invention concerns a method for separating, by impaction, living particles and particles capable of being revived of a gas or a gas mixture under pressure, characterised in that it comprises a step (a) which consists in accelerating in a stage and along an axis (x) a single gas or gas mixture stream in a confined volume (V), to reach a speed not less than the speed of sound; a step (b) which consists in sudden deceleration of said single stream derived from step (a) in said volume, along substantially the same axis (x), at atmospheric pressure; a step (c) which consists in changing the direction of the gas or gas mixture flow at the output of the volume (V), which on average becomes radial; and a step (d) which consists in trapping said particles separated from the flow during step (c), on a target. In another embodiment, the method further comprises a step (e) which consists in counting the particles trapped during step (d). The invention also concerns a method for determining the microbiological quality of a gas under pressure, characterised in that it consists in using said alternative embodiment. The invention further concerns a device (D) designed to implement said method and its alternative embodiment comprising an element (D₁) wherein the single gas stream is successively subjected to steps (a) and (b), an element (D₂) constituting the target whereon the living particles or particles capable of being revived are trapped as in step (d), and securing means for making (D₁) and (D₂) integral, while allowing the flow of gas or of gas mixture to change direction as in step (c). The device is useful for determining the microbiological quality of a gas under pressure.

The invention relates to the field of the production, applications and analysis of gases.

The bacteriological quality of gases, and in particular air, is becoming a necessity. There is commercial equipment which makes it possible to monitor the microbiological quality of ambient air. This equipment is used in hospitals, pharmaceutical laboratories, in the food industry and in any activity which needs to be carried out in “clean rooms”, such as the fabrication of electronic components. This equipment operates according to two methods: the filtration method (MILLIPORE™ equipment and SARTORIUST™ equipment) and the impact method.

The filtration method consists in passing a gas through a filter holder fitted with a filter whose pore size is between about 0.22 μm and 0.45 μm. The microorganisms which are being carried by the gas flow and whose size is greater than that of the pores are retained by the filter. The filter is subsequently collected under aseptic conditions, and it is either washed in order to recover the microorganisms or put directly into culture on a Petri dish. After a specified period of incubation in the oven, the number of colony-forming units (CFU) is counted and the germs that are present are identified. The number of colony-forming units per volume of sampled gas, expressed in CFU/m³, is deduced therefrom. In a variant of this method, a water-soluble filter based on gelatin is used which makes it possible, after having added water, to put the filter directly into culture or alternatively deposit it on a Petri dish.

The impact method consists in sampling the gas, accelerating its flow strongly and directing it at a target coated with gelose. The particles and microorganisms which have sufficient momentum leave the air flow and are thrown onto the surface of the gelose-coated target. There are two variants of this method: “screen impacting”, according to which the gas passes through a plate pierced with holes. Each hole initiating a gas jet which impacts the target (AIR TEST™ OMEGA equipment), and “impact by centrifuging” or by “rotation of the flow” according to which the particles are separated from the gas flow in order to be thrown by centrifuging onto a strip of gelose (Biotest's RCS High Flow™ equipment or ECOMESURE's BIAP™ equipment).

For the microbiological control monitoring of ambient air, the majority of equipment operates according to the impact method. However, this equipment does not allow the contamination of the air to be fully determined.

There is also equipment which operates according to the same method for monitoring the quality of compressed gases, for example the BIO-IMPACTOR™ from AIR STRATEGIE™. The aforementioned equipment is designed to operate at the pressure of the network to which it is connected, with a constant gas flow rate of 28 liters/minute. However, the results obtained with this equipment are not very reproducible because they depend on a large number of rates, such as the size of the viable particles to be analyzed, the type and sensitivity of the microorganisms, the contamination levels, the environmental conditions, the accuracy and efficiency of the sampling by the operator, the type and height of the gelose covering the target, the impact velocity of the particles and the volume of gas sampled, via its capacity to dry the gelose to a greater or lesser extent. This is why the results for a given gas vary from one equipment unit to another, irrespective of whether they are identical or different.

Furthermore, although the pumps and the suction systems of this equipment are calibrated and monitored so as to sample a precise volume of gas at a given time with a constant velocity, there is no calibration relationship for precisely determining the recovery this equipment are not very reproducible because they depend on a large number of rates, such as the size of the viable particles to be analyzed, the type and sensitivity of the microorganisms, the contamination levels, the environmental conditions, the accuracy and efficiency of the sampling by the operator, the type and height of the gelose covering the target, the impact velocity of the particles and the volume of gas sampled, via its capacity to dry the gelose to a greater or lesser extent. This is why the results for a given gas vary from one equipment unit to another, irrespective of whether they are identical or different.

Furthermore, although the pumps and the suction so as to sample a precise volume of gas at a given time with a constant velocity, there is no calibration relationship for precisely determining the recovery rate of the living microorganisms actually present in the volume the air analyzed. Certain suppliers have defined the recovery rates as a function of the size of the particles, but the biological efficiency, which represents the capacity of the collector to maintain the viability of the microorganisms being collected throughout the sampling procedure, has been validated only little or not at all.

Lastly, there is currently no standard defining a maximum number of microorganisms that must not be exceeded per m³ of ambient air or per m³ of analyzed gas, whether in the food industry, in the pharmaceutical industry or even in the medical field. In the absence of any standards specific to aero-biocontamination (with the exception of clean rooms), it is necessary to establish mapping of this contamination. The only standards that exist relate to dustiness.

The Applicant has therefore sought to develop a method for separating particles from a gas or a gas mixture.

This is the reason why the invention relates to a method for impact-separation of living or revivable particles from a gas or a gas mixture under pressure containing them, characterized in that it comprises:

a step (a) of accelerating, in one stage and along an axis (x), a monoflow of the gas or the gas mixture in a confined volume (V), in order to reach a velocity greater than or equal to the speed of sound,

a step (b) of sharply decelerating said monoflow coming from step (a) in said volume, substantially along the same axis (x), to atmospheric pressure,

a step (c) of changing the direction of the stream of the gas or the gas mixture at the exit of the volume (V), which becomes radial on average, and

a step (d) of trapping said particles separated from the stream during step (c), on a target.

The term gas or gas mixture under pressure denotes a gas or a gas mixture at a total pressure of more than 1 atmosphere (about 10⁵ Pa), generally at a total pressure of less than or equal to 200 atmospheres, and more particularly at a total pressure of less than or equal to 10 atmospheres.

The term sharp in the present patent application is intended to mean a deceleration which makes it possible to reduce the velocity of a gas stream, having a Mach number of between 1 and 3, to a zero axial velocity on the target.

The term confined volume (V), in which steps (a) and (b) are carried out and at the end of which step (c) is carried out, is intended in the present invention to mean a finite volume delimited by a finite lateral surface, and said lateral surface is a surface of revolution about the axis (x).

The flow rate of gas whose microbiological quality is intended to be determined varies depending on the origin and/or the use which is made of said gas. It is often between 50 liters per minute and 400 liters per minute, and more particularly between 100 liters per minute and 200 liters per minute.

The method to which the present invention relates can be carried out with any gas or gas mixture.

As examples of gases or gas mixtures, there are for example air, oxygen (O₂), nitrogen (N₂), carbon dioxide (CO₂), helium (He), nitrous oxide (N₂O), nitrogen monoxide (NO), mixtures of nitrous oxide and oxygen, carbon dioxide and oxygen, nitrogen and nitrogen monoxide and nitrogen monoxide or helium and oxygen, and more particularly mixtures (50% by volume (v/v) N₂O+50% v/v O₂), (5% v/v CO₂+95% v/v O₂), (200 ppm to 800 ppm NO in N₂), (78% v/v He+22% v/v O₂), (65% He+35% O₂) and (80% v/v He+20% v/v O₂), used in medicine or mixtures of nitrogen and carbon dioxide (N₂+CO₂) used in the food industry.

According to a first particular aspect, the confined volume consists of a first volume fraction (Va), in which step (a) is carried out and which is delimited by two bases (B₁) and (B₂) of areas S₁ and S₂, with S₁ greater than or equal to S₂, and a second volume fraction (V_{b}) contiguous to (Vₐ), in which step (b) is carried out and which is delimited by (B₂) and a base (B₃) of areas S₃, with S₃ greater than or equal to S₂.

When S₁ and S₂ are equal, S₁ is less than the cross section of the gas flow circulating upstream of the volume (V).

According to a particular version of the method to which the present invention relates, the velocity of the flow of the gas or the gas mixture coming from step (a) is greater than the speed of sound.

According to another particular version of the method to which the present invention relates, the axial velocity along the axis (x) of the flow of the gas or the gas mixture coming from step (b) tends to zero.

According to another particular version of the method as defined above, the trapping step (d) is carried out on a target surface capable of fixing said particles.

The term living and revivable particles denotes microorganisms of any orders, which are capable of reproducing under suitable pressure and temperature conditions.

Preferably, the target surface is that of a nutrient medium allowing development and reproduction of the trapped microorganisms. It may be a liquid medium or a solid medium such as gelose. Depending on the type of microorganisms whose presence is intended to be determined, said medium will be specific to the growth of one or more bacterial species or non-specific.

As examples of solid nutrient a medium employed for trapping the particles, there are:

the medium TCS™ (AES laboratory, reference: AEB 522 859) (tryptocasein soy), which is a non-selective medium comprising 15 g/l of pastone, 5 g/l of soy papainic peptone, 5 g/l of sodium chloride and 15 g/l of agar;

the medium R2A™ (AES laboratory, reference AEB 523 480) which is a non-selective medium comprising 1.0 g/l of a mixture of peptones (peptone proteose), 0.5 g/l of yeast extract, 0.5 g/l of casein acid hydrolysate, 0.5 g/l of dextrose, 0.5 g/l soluble starch, 0.3 g/l of dipotassium phosphate (K₂HPO₄), 0.024 g/l of magnesium sulfate, 0.3 g/l of sodium pyruvate and 15 g/l of agar.

the medium R3A™ which is a non-selective medium comprising 1.0 g/l of a mixture of peptones (peptone proteose), 1.0 g/l of yeast extract, 1.0 g/l of casein acid hydrolysate, 1.0 g/l of dextrose, 1.0 g/l of soluble starch, 0.6 g/l of dipotassium phosphate (K₂HPO₄), 0.10 g/l of magnesium sulfate, 0.10 g/l of sodium pyruvate and 30 g/l of agar.

The MOSSEL medium (AES laboratory, reference AEB 521 740) which is a medium specific to the growth of Bacillus cereus and Bacillus spp in general.

The impact hardness of these media can be modified by modifying the concentration of agar or agar-agar.

The invention also relates to a variant of the method as defined above, furthermore comprising a step (e) of counting the particles trapped during step (d).

Step (e) preferably consists of a step e₁ of culturing the trapped living and revivable particles coming from step (d), followed by a step (e₂) of counting the colony-forming units. The culture step may consist in keeping some of the nutrient material containing the trapped particles at a temperature allowing reproduction of the microorganisms for a specified time.

The method as defined above makes it possible to efficiently separate microorganisms having dimensions of between 0.05 μm and 25 μm, and more particularly between 0.2 μm and 12 μm.

As examples of microorganisms whose concentration in a gas under pressure was determined by the variant of the method as described above, there are mold fungi and yeasts Aspergillus candidus, Aspergilus nidulans, Aspergillus niger, Blastomyces,dermatitis, Mucor indicus, Penicillum glarum, Rhodotorula lubra, Cryptococcus albidus, hansenula polymorpha, Rhodotorula mucilaginosa, Rhodotorula mucilaginosa or Candida kefyr; Gram⁺ bacteria such as Brevibacillus brevis, Bacillus sphaericus, Bacillus Cereus, Bacillus anthracis, Bacillus subtilis, Bacillus coagulans, Micrococcus luteus, Micrococcus varians, Staphylococcus xylosus, Staphylococcus haemolyticus, Staphylococcus saprophyticus, Staphylococcus hominis or Staphylococcus warneri; Gram⁻ bacteria such as Acinetobacter sp, Escherichia vulneris, Pseudomonas chloraphis, Pseudomonas,vesicularis or Chryseomonas luteola.

According to another object of the present invention, it relates to a method for determining the microbiological quality of a gas or a gas mixture under pressure by carrying out the variant of the separation method as described above.

The method and its variant make it possible for germs present in a gas under pressure to be collected on a Petri dish. The gas being analyzed may come from bottles, surroundings or a network (primary or secondary), and more particularly a network for supplying medical gases such as are found in healthcare establishments such as hospitals, dispensaries or clinics. The gas does not need to have its pressure reduced to atmospheric pressure in order to be analyzed by the method described above.

According to another aspect of the present invention, it relates to a device (D) suitable for carrying out the method and its variant as defined above.

The device (D) comprises:

An element (D₁) in which the gas monoflow successively undergoes steps (a) and (b),

An element (D₂) constituting the target on which the living or revivable particles are trapped according to step (d), and

Means for joining (D₁) and (D₂) together while allowing the stream of the gas or the gas mixture to change direction according to step (c).

The element (D₁) of the device (D) is a hollow solid, comprising an entry orifice, an exit orifice and an inner lateral surface S_{L}, defining a confined volume (V) having polygonal, elliptical or cylindrical orthogonal sections, in which steps (a) and (b) take place and at the exit orifice of which step (c) takes place. The surface S_{L} is more particularly either a surface of revolution about an axis (x), or a lateral surface of a regular polyhedron with a symmetrical axis (x), or a set of one or more surfaces of revolution and/or one or more surfaces of regular polyhedra, which have the same symmetry axis (x), defining the volume (V). The two orifices are preferably coaxial with respect to the axis (x).

The element (D₁) may be made of any material, whether a plastic a metal or an alloy of metals. it is preferably made of a heat-sterilizable material.

The element (D₁) of the device (D) more particularly comprises an internal lateral surface SL defining a first volume fraction (Vₐ) of frustoconical or cylindrical shape, in which step (a) is carried out, and a second volume fraction (V_{b}), contiguous to (Vₐ) and of frustoconical shape, in which step (b) is carried out.

The element (D₁) of the device (D) especially has a shape defining a first fraction of cylindrical shape with a height h and a diameter w, in which step (a) is carried out, and a second fraction of frustoconical shape with a height H, having a small base with a diameter w and a large base with a diameter W, in which step (b) is carried out. The entry orifice in this case has a circular cross section with a diameter w and the exit orifice has a circular cross section with a diameter W.

The ratio h/w is in general between 10/1 and 1/10. It is more particularly between 10/2 and 2/10.

The ratio W/w is in general between 50/1 and 5/1. It is more particularly between 25/1 and 10/1.

The ratio H/w is in general between 5/1 and 50/1. It is more particularly between 10/1 and 40/1.

H is in general between 10 mm and 200 mm, more particularly between 30 mm and 120 mm and especially between 40 mm and 100 mm

When the total pressure of the gas entering the device is less than 5 atmospheres, in the element (D₁) of the device (D) used for carrying out the method as described above, the value of w is in general between about 2 mm and 5 mm, and it is more particularly equal to about 4 mm.

The element (D₁) as defined above also constitutes another aspect of the present invention.

The element (D₂) of the device (D) is preferably of cylindrical shape and has a diameter W₁ greater than or equal to W. In general, W₁ is less than or equal to 90 mm. W is in general between 50 mm and 90 mm. It is preferably a Petri dish. The element (D₂) contains in general a nutrient medium for the microorganisms, and more particularly gelose.

The means for joining D₁ and D₂ together while allowing the stream of the gas or the gas mixture to change direction according to step (c) of the method as defined above are, for example, studs. They make it possible to hold the element D₂ at a distance d from D₁ which is sufficient to allow the entire stream of the gas or the gas mixture to the exit of the element D₁, on average radially with respect to the axis (x).

The ratio H/d is in general between 2/1 and 20/1. It is more particularly between 3/1 and 15/1.

The studs preferably make it possible to release the elements D₁ and D₂, in order to carry out the culture of the living and revivable particles more easily.

The device (D) as defined above may furthermore comprise a connection element (D₀), for example a serrated plug, capable of being fitted to a medical gas outlet point, a tube with a small diameter capable of being fitted to the exit of a pressure reducer and, more generally, any connector capable of being fitted to any pressurized gas delivery device.

The device (D) as defined above may furthermore comprise a pressure reducer placed between the connection element (D₀) and the element (D₁).

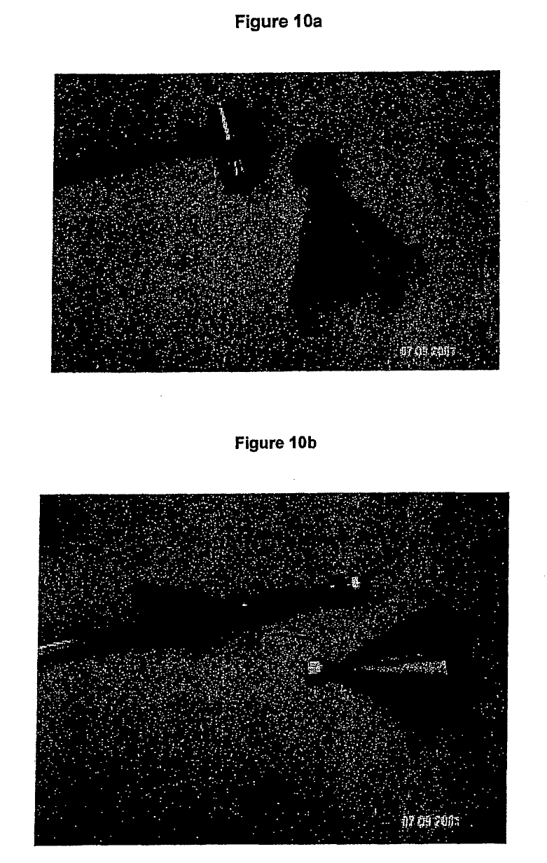

The device (D) as defined above may be connected to the supply of gas to be analyzed either by being connected to a wall outlet point perpendicularly to the pipe, or by being connected coaxially to the supply of the gas to be analyzed, preferably downstream of a ¼ turn valve. such options are illustrated by FIGS. 10a and 10b.

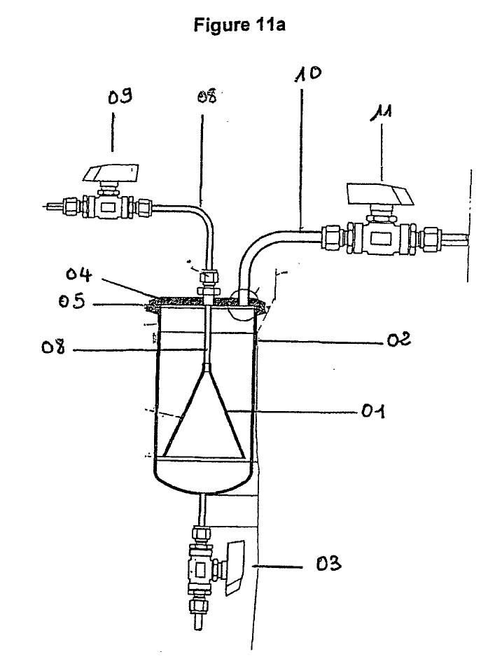

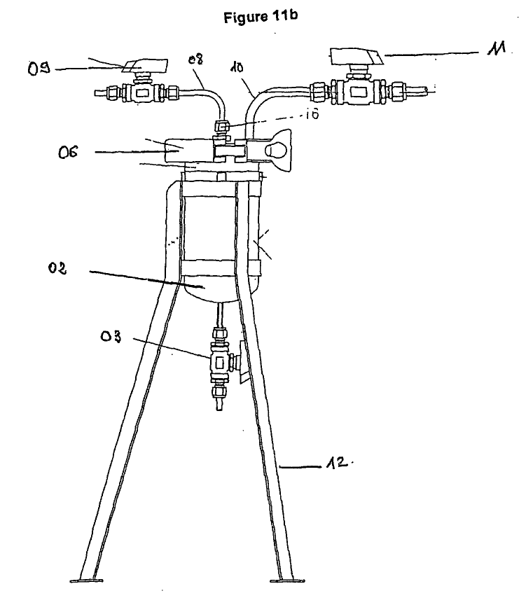

When the target surface is a liquid medium such as water or a nutrient solution, element D₁ is placed in a collection vessel as represented in FIG. 11a. In this figure, element D₁ is the element (01).

The vessel (02) is provided with a drainage valve (03) in its lower part, which makes it possible to collect the liquid containing the impacted particles.

It is closed in its upper part by a lid (04), if necessary provided with a seal (05), for example by means of a clamping collar (06). The lid (04) is pierced at its center so as to permit communication between the element (01) and a valve (09) by using a tube (08). It is also pierced at an off-center position so as to connect it to an outlet valve (11) via a tube (10).

According to a particular aspect of this variant, the collection vessel as well as the lid are made of metal and can be sterilized in an autoclave.

According to another particular aspect, the vessel has a capacity of about one liter, and it withstands a maximum pressure of about 5.5 atmospheres.

According to another aspect of the present invention, the position of the element (01) in the vessel (02) is adjustable in height so as to be able to modify the quantity of liquid or the impact height in relation to the upper surface of the liquid.

The vessel may rest on a tripod (12), as illustrated by FIG. 11.

According to another aspect of the present invention, it relates to the use of the device (D) or the element (D₁) for determining the microbiological quality of a gas or a gas mixture under pressure, or for determining the microbiological quality of the atmosphere of rooms.

The method and the device to which the present invention relates can be employed for determining the microbiological quality of gases or gas mixtures contained in bottles, whether these are bottles of medical gases or industrial bottles such as those intended for the electronic-component fabrication industry, the food industry or the pharmaceutical industry.

The method and the device to which the present invention relates can also be employed for determining the microbiological quality of gases or gas mixtures delivered by supply networks, such as hospital supply networks, in particular for delivering gases into the operating theaters or units, the supply networks of deep-frozen food production lines, networks for supplying gases with very high purity for the fabrication of electronic components, or networks for supplying gases needed for the manufacture and/or packaging of pharmaceutical products and formulations

The method and the device to which the present invention relates can also be employed for determining the microbiological quality of gases or gas mixtures at the exit of a unit for producing the gas or the gas mixture, for example a compressor, a cryogenic distillation column, a column for separating gases by adsorption, whether a PSA (pressure swing adsorption) column, a VSA (volume swing adsorption) column or a TSA (temperature swing adsorption) column, or a unit for separating gases by permeation, through polymer membranes, for example a FLOXAL™ unit.

The method and the device to which the present invention relates can also be employed for determining the microbiological quality of the ambient air in rooms, and more particularly in clean rooms of sites for manufacturing medicaments or sites for fabricating electronic components, or alternatively sites for storing documents.

The following examples illustrate the invention, but without limiting it.

### EXAMPLE OF A DEVICE ACCORDING TO THE INVENTION

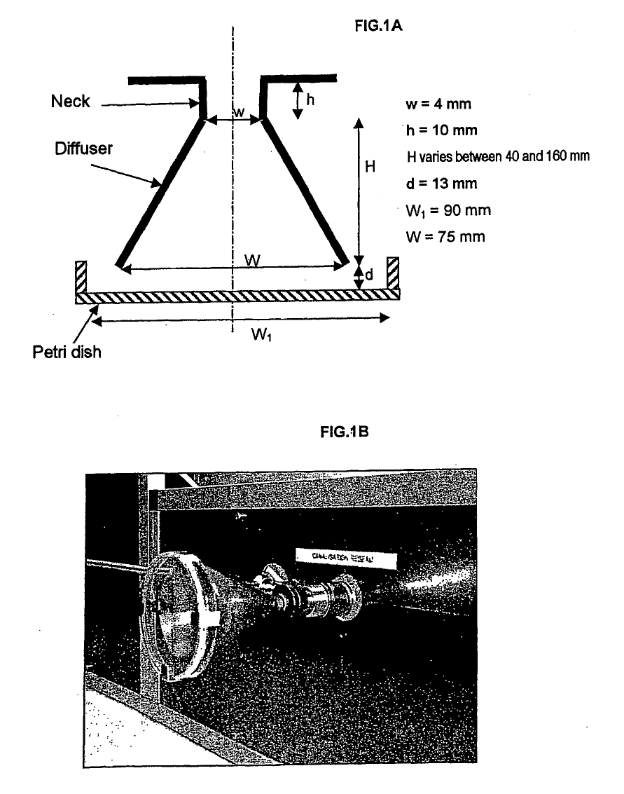

FIG. 1A illustrates a device according to the invention. It consists of a neck, a diffuser and a Petri dish. The device operates so that the velocity of the jet continues to increase, so long as its pressure has not reached ambient pressure. The total supply pressure is 3.5 bar. The total temperature is 300K.

A device for which H is equal to 80 mm is presented on the photograph in FIG. 1B.

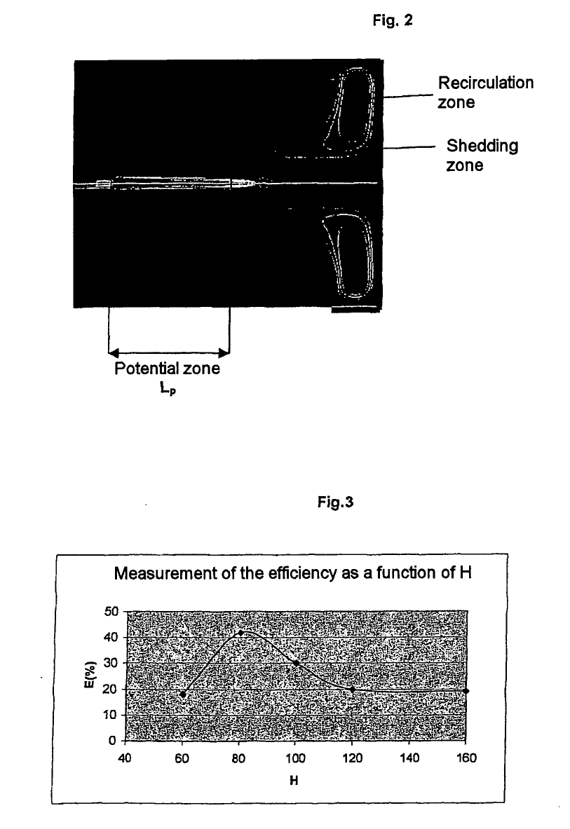

FIG. 2 demonstrates the velocity fields in the device according to the invention. In the neck, the fluid will accelerate up to the speed of sound at the exit of the neck, then there are zones of pressure/pressure reduction to a minor extent, where the stream will be alternately accelerated and slowed. This zone is defined here as a potential zone of length Lₚ, turbulence not playing a part. A number of simulations were carried out for diffuser lengths of 40 mm, 60 mm, 80 mm, 100 mm and 120 mm. Likewise, experimental measurements of impact efficiency were carried out for values of 60 mm, 80 mm, 100 mm, 120 mm and 160 mm.

A new formulation of the Stockes number is defined for this type of impactor by the formula:

$$St = {CSt}_{0} \frac{w}{H - {L}_{p}}$$

with C between 1 and 5 and Lₚ being the length of the potential zone. Lₚ can be calculated for this example by the formula:

$$\frac{{L}_{p}}{{d}_{j}} = 4.2 + 1.1 ( {M}_{j}^{2} + 1 - \frac{{T}_{j}}{{T}_{a}} )$$

in which the index j corresponds to the state after isentropic expansion from the generating pressure to the atmospheric pressure.

It constitutes a generalization of the empirical formula given by Lau, Morris and Fisher:

$${St}_{0} = {τ}_{0} \frac{{V}_{j max}}{w}$$

with V_{j max} being the maximum velocity of the fully developed jet,

τ=dₚ²ρₚK/(9μ)

K being between 0.1 and 2, p being the diameter of the particles, ρₚ being the density of these particles. μ The viscosity of the fluid carrying said particles.

H is calculated such that \sqrt{St}>0.2.

FIG. 3 represents the curve of efficiency of the device according to the invention, as a function of H, d being fixed at 13 mm.

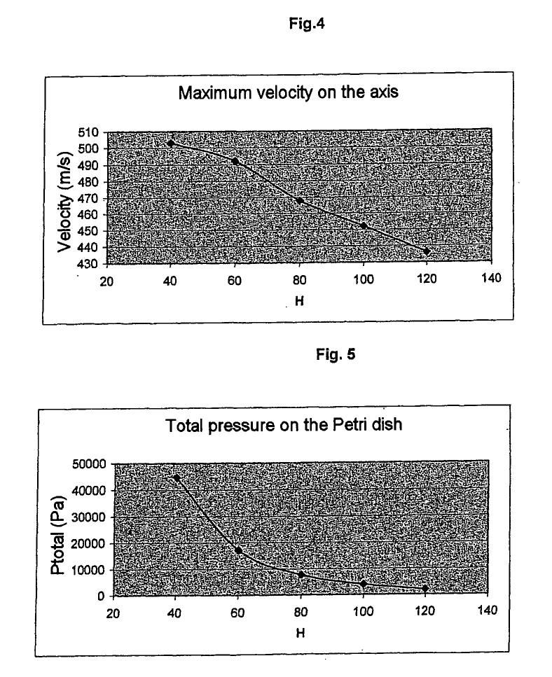

FIG. 4 represents the curve of the maximum axial velocity of the monoflow in the device according to the invention has a function of H.

FIG. 5 represents the curve of the total pressure on the target in the device according to the invention as a function of H

For the total pressure, in order to preserve the integrity of the gelose being used in this particular application, it was desirable to have Pt<7500 Pa, which implies H=80 mm for the device in FIG. 1B.

Determination of the Bacteriological Quality of Gas.

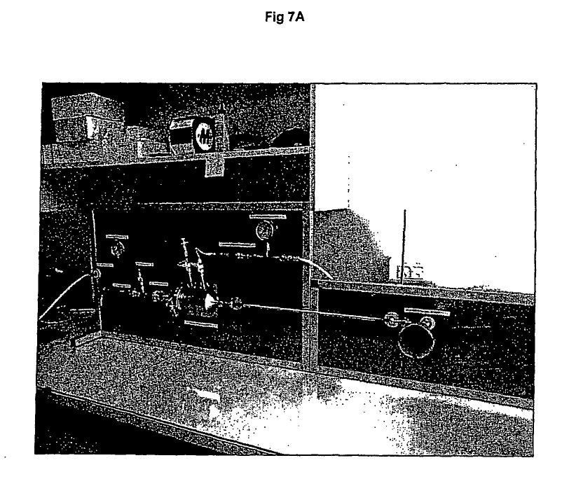

The test bench presented in the photograph of FIG. 7A, fitted with the device according to the invention, is used. This test bench is connected directly to a network outlet point or to the exit of a pressure reducer. The device presented in FIG. 1A is connected to the end of the test bench.

Sampling is carried out for a specified time, which corresponds to a perfectly known quantity of gas being analyzed (generally 100L).

The Petri dish which was used for collecting the germs is then placed in the oven.

The parameters of microorganism cultures (choice of the culture medium, temperature and incubation time) are selected as a function of the type of germs to be identified. The Petri dishes on which the particles contained in the gas flow have impacted are put into culture. After incubation, the number of CFUs is counted and this number is subsequently normalized to the analyzed gas volume. This makes it possible to determine a number of CFUs/m³ of gas.

Each colony can then be transplanted for identification.

As a function of the pressure of the gas to be analyzed, a sampling type is determined which in general lies between 10 seconds and 3 minutes, so that the volume of gas to be analyzed is sufficient and in general less than 500L.

A known volume of a suspension of bacteria in a specified quantity is sprayed while a defined volume of gas is being injected into the test bench. At the exit of the bench, a known volume of gas is therefore available which contains a perfectly determined number of microorganisms suspended in this volume. The bacteria are collected at the exit of the pipe by using the device according to the invention. Knowing the quantity of microorganisms injected and the quantity collected by the collector, the recovery rate of the device can be deduced therefrom. A plurality of cone lengths were tested:

The cone with a length of 50 mm gives a recovery rate of 20%

The cone with a length of 80 mm gives a recovery rate of 40%

The cone with a length of 170 mm gives a recovery rate of 20%

We also studied the entry diameter of the cone.

We tested diameters of 2 mm and 4 mm.

The 4 mm diameter (for length of 80 mm) gives a recovery rate of 40%

The 2 mm diameter (for length of 80 mm) gives a recovery rate of 12%

It is therefore a cone with a length of 80 mm and a 4 mm orifice which gives the best recovery rate.

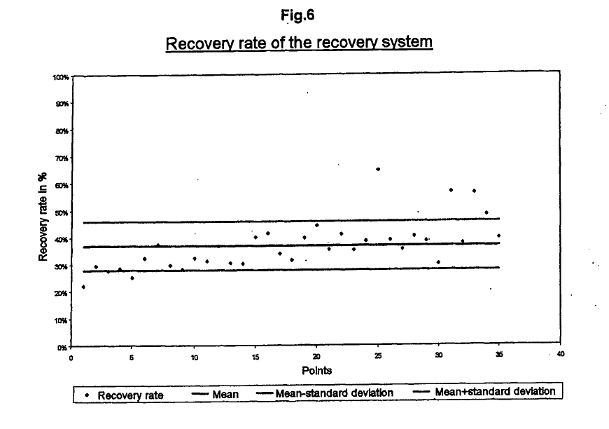

A series of 35 measurements is carried out. The recovery rate is 40%. Most of the points (88%) lie between +1 standard deviation and −1 standard deviation. The results are represented on the curve in FIG. 6.

These results highlight the advantages of the device according to the invention: It limits the entrainment of ambient air that pollutes the gas, it makes it possible to obtain very sharp deceleration of the gas flow, it limits the total pressure on the gelose to a value of the order 7500 Pa, and it avoids having re-entrainment of the microorganisms by limiting the shear levels on the gelose to 80 Pa.

Recovery rates are obtained which are centered around 40% and are reproducible; this constitutes a result which is far superior to the results described in the prior art, which rarely exceed 20% and do so non-reproducibly.

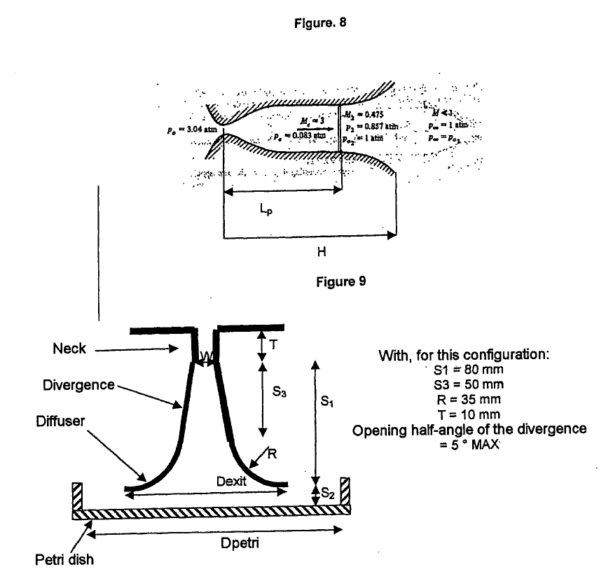

FIGS. 8 and 9 represent devices according to the invention having non-frustoconical axisymmetric shapes. In the dimensioning of the device in FIG. 8, the distance between the neck and the normal impact represents Lₚ. H is determined such that \sqrt{St}>0.2.

During a final series of measurements, we studied the effect of the flow rate of gas to be analyzed with a device (D) having an entry diameter of 4 mm and length of H=80 mm, connected to a 6/1 mm gas supply tube.

The results obtained are reported in the table below:

| Flow rate of gas in liters/minute | Number of CFUs |
|---|---|
| 76 | 47 |
| 95 | 76 |
| 160 | 78 |
| 199 | 82 |

1. A method for impact-separation of living or revivable particles from a gas or a gas mixture under pressure containing them, characterized in that it comprises:
A step (a) of accelerating, in one stage and along an axis (x), a monoflow of the gas or the gas mixture in a confined volume (V), in order to reach a velocity greater than or equal to the speed of sound,
A step (b) of sharply decelerating said monoflow coming from step (a) in said volume, substantially along the same axis (x), to atmospheric pressure,
A step (c) of changing the direction of the stream of the gas or the gas mixture at the exit of the volume (V), which becomes radial on average, and
A step (d) of trapping said particles separated from the stream during step (c), on a target.

2. The method as defined in claim 1, in which the volume (V) consists of a first volume fraction (Vₐ), in which step (a) is carried out and which is delimited by two bases (B₁) and (B₂) of areas S₁ and S₂, with S₁ greater than or equal to S₂, and a second volume fraction (V_{b}) contiguous to (Vₐ), in which step (b) is carried out and which is delimited by (B₂) and a base (B₃) of area S₃, with S₃ greater than or equal to S₂.

3. The method as defined in claim 2, for which, when S₁ and S₂ are equal, S₁ is less than the cross section of the gas flow circulating upstream of the volume (V).

4. The method as defined in any one of claims 1 to 3, in which the velocity of the flow of the gas or the gas mixture coming from step (a) is greater than the speed of sound.

5. The method as defined in any one of claims 1 to 4, in which the axial velocity along the axis (x) of the flow of the gas or the gas mixture coming from step (b) tends to zero.

6. The method as defined in any one of claims 1 to 5, in which the trapping step (d) is carried out on a target surface capable of fixing said particles.

7. A variant of the method as defined in any one of claims 1 to 6, furthermore comprising a step (e) of counting the particles trapped during step (d).

8. A variant of the method as defined in claim 7, in which step (e) consists of a step e₁ of culturing the trapped living and revivable particles coming from step (d), followed by a step (e₂) of counting the colony-forming units.

9. A method for determining the microbiological quality of a gas or a gas mixture under pressure, which carries out the variant of the method as defined in one of claims 7 and 8.

10. A device (D) suitable for carrying out the method and its variant as defined in any one of claims 1 to 8, comprising:
An element (D₁) in which the gas monoflow successively undergoes steps (a) and (b),
An element (D₂) constituting the target on which the living or revivable particles are trapped according to step (d), and
Means for joining (D₁) and (D₂) together while allowing the stream of the gas or the gas mixture to change direction according to step (c).

11. The device as defined in claim 10, in which the element (D₁) is a hollow solid, comprising an entry orifice, an exit orifice and an inner lateral surface S_{L}, defining a confined volume (V) having polygonal, elliptical or cylindrical orthogonal sections, in which steps (a) and (b) take place and at the exit orifice of which step (c) takes place.

12. The device as defined in claim 11, in which the surface S_{L} is either a surface of revolution about an axis (x), or a lateral surface of a regular polyhedron with a symmetrical axis (x), or a set of one or more surfaces of revolution and/or one or more lateral surfaces of regular polyhedra, which have the same symmetry axis (x) and in which the entry and exit orifices are coaxial along the axis (x).

13. The device as defined in claim 12, in which the lateral surface S_{L} defines a first volume fraction (Vₐ) of frustoconical or cylindrical shape, in which step (a) is carried out, and a second volume fraction (V_{b}), contiguous to (Vₐ) and of frustoconical shape, in which step (b) is carried out.

14. The device as defined in claim 13, in which the element (D₁) has a shape defining a first fraction of cylindrical shape with a height h and a diameter w, and a second fraction of frustoconical shape with a height H, having a small base with a diameter w and a large base with a diameter W, the entry orifice having a circular cross section with a diameter w and the exit orifice having a circular cross section with a diameter W.

15. The device as defined in claim 14, in which the ratio h/w is between 10/1 and 1/10, and more particularly between 10/2 and 2/10.

16. The device as defined in either one of claims 14 and 15, in which the ratio W/w is between 50/1 and 5/1, and more particularly between 25/1 and 10/1.

17. The device as defined in one of claims 14 to 16, in which the ratio H/w is between 5/1 and 50/1, and more particularly between 10/1 and 40/1.

18. The device as defined in one of claims 14 to 17, in which H is between 10 mm and 200 mm, more particularly between 30 mm and 120 mm and especially between 40 mm and 100 mm.

19. The device as defined in any one of claims 14 to 18, in which w is between about 2 mm and 5 mm, and more particularly in which w is equal to 4 mm.

20. An element (D₁) as defined in any one of claims 10 to 19.

21. The device as defined in one of claims 14 to 19, in which the element (D₂) of the device (D) is of cylindrical shape and has a diameter W₁ greater than or equal to W, and is more particularly a Petri dish.

22. The device as defined in any one of claims 14 to 19, in which the element (D₂) contains a nutrient medium for the microorganisms, and more particularly gelose.

23. The device as defined in any one of claims 14 to 19 and 21 to 22, in which the means for joining (D₁) and (D₂) together while allowing the stream of the gas or the gas mixture to change direction according to step (c) hold the element (D₂) at a distance d from (D₁) which is sufficient to allow the entire stream of the gas or the gas mixture to the exit of the element (D₁), on average radially with respect to the axis (x), in a ratio H/d of between 2/1 and 20/1, and more particularly between 3/1 and 15/1.

24. The device as defined in any one of claims 14 to 19 and 21 to 23, in which furthermore comprising a connection element (D₀) capable of being fitted to any pressurized gas delivery device.

25. The device as defined in any one of claims 14 to 19 and 21 to 24, furthermore comprising a pressure reducer placed between the connection element (D₀) and the element (D₁).

26. The device as defined in any one of claims 10 to 19 and 24 and 25, in which the element D₁ (01) is placed in a vessel (02).

27. The device as defined in claim 26, in which the vessel (02) is provided with a valve (03) in its lower part and is closed by a lid (04) in its upper part, said lid (04) being pierced at its center so as to permit communication between the element (01) and a valve (09) by using a tube (08); and said lid (04) being pierced at an off-center position so as to connect it to an outlet valve (11) via a tube (10).

28. Use of the device as defined in any one of claims 10 to 19 and 21 to 27, for determining the microbiological quality of a gas or a gas mixture under pressure.

29. Use of the element (D₁) as defined in claim 20 for determining the microbiological quality of a gas or a gas mixture under pressure.

30. Use of the device as defined in any one of claims 10 to 19 and 21 to 27 for determining the microbiological quality of the atmosphere of rooms.

31. Use of the element (D₁) as defined in claim 20 for determining the microbiological quality of the atmosphere of rooms.

32. Application of the method and its variant as defined in one of claims 1 to 9 for determining the microbiological quality of gases or gas mixtures contained in bottles, and more particularly bottles of medical gases or gases intended for the electronic-component fabrication industry, the food industry or the pharmaceutical industry.

33. Application of the method and its variant as defined in one of claims 1 to 9 for determining the microbiological quality of gases or gas mixtures delivered by supply networks, and more particularly by hospital supply networks, in particular for delivering gases into the operating theaters or units, supply networks of deep-frozen food production lines, networks for supplying gases with very high purity for the fabrication of electronic components, or networks for supplying gases needed for the manufacture and/or packaging of pharmaceutical products and formulations.

34. Application of the method and its variant as defined in one of claims 1 to 9 for determining the microbiological quality of gases or gas mixtures at the exit of a unit for producing the gas or the gas mixture, and more particularly at the exit of a compressor, a cryogenic distillation column, a column for separating gases by adsorption or a unit for separating gases by permeation, through polymer membranes.

35. Application of the method and its variant as defined in one of claims 1 to 9 for determining the microbiological quality of the ambient air in rooms, and more particularly in clean rooms of sites for manufacturing medicaments or sites for fabricating electronic components, or alternatively sites for storing documents.
